(19) United States
(12) Patent Application Publication
(10) Pub. No.: US 2014/0323924 A1
(43) Pub. Date: Oct. 30, 2014

(54) TARGETED OPTOGENETIC NEUROMODULATION FOR TREATMENT OF CLINICAL CONDITIONS

(71) Applicant: David J. Mishelevich, Playa del Rey, CA (US)

(72) Inventor: David J. Mishelevich, Playa del Rey, CA (US)

(21) Appl. No.: 13/871,237

(22) Filed: Apr. 26, 2013

(51) Int. Cl. A61K 38/17; A61N 7/00; A61N 5/06; A61K 41/00

(52) U.S. Cl. CPC A61K 38/177; A61K 41/00; A61N 7/00; A61N 5/062
USPC 601/2; 604/20

(57) ABSTRACT

Disclosed are methods and systems and methods for methods for neuromodulation of deep-brain and other neural targets in mammals using optogenetics to treat clinical conditions or achievement of a physiological state. The neuromodulation can produce acute or long-term effects. The latter occur through Long-Term Depression (LTD) and Long-Term Potentiation (LTP) via training. Included is control of optical intensity/amplitude, pulse width, pulse shape, pulse rate, burst frequency, pulse pattern, burst rate, burst width, and optical-fiber configuration including through the stimulation of incorporated opsins in the target neural membranes accomplishing up-regulation and/or down-regulation.

### CROSS REFERENCE TO RELATED APPLICATIONS

This patent application claims priority to Provisional Patent Application No. 61/638,497 filed Apr. 26, 2012, entitled “TARGETED OPTOGENETIC NEUROMODULATION FOR TREATMENT OF CLINICAL CONDITIONS.”

### INCORPORATION BY REFERENCE

All publications, including patents and patent applications, mentioned in this specification are herein incorporated by reference in their entirety to the same extent as if each individual publication was specifically and individually cited to be incorporated by reference.

### FIELD OF THE INVENTION

Described herein are systems and methods for Optogenetic Neuromodulation including one or more neuromodulated neural-structure targets in deep-brain or superficial regions to up-regulate or down-regulate neural activity for the treatment of a medical condition or attainment of a physiological state.

### BACKGROUND OF THE INVENTION

It has been demonstrated that optogenetic stimulation directed at neural structures can neuromodulate those structures. If neural activity is increased or excited, the neural structure is up regulated; if neural activated is decreased or inhibited, the neural structure is down regulated. Neural structures are usually assembled in circuits. For example, nuclei and tracts connecting them make up a circuit.

Several patent applications have dealt with the mechanism for accomplishing optogenetic neuromodulation. They are:

Schneider, M B, Mishelevich, D J, and K Deisseroth, “System for Optical Stimulation of Target Cells,” U.S. patent application Ser. No. 11/651,422 filed 2007-01-09, Publication Number US 2008/0085256, 2008-04-10.
Zhang, F., Deisseroth, K., Mishelevich, D J, and M B Schneider, “System for Optical Stimulation of Target Cells,” International Application Number: PCT/US2008/050628 filed 2008-01-09, WO 2008/089003, 2008-07-28.
Aravanis, A, Deisseroth, K, Zhang, F., Schnieder, M B, and J M Henderson, “Optical Tissue Interface Method and Apparatus for Stimulating Cells,” U.S. patent application Ser. No. 12/185,624, filed 2008-08-04, Publication Number US 2009/0088680, 2009-04-02.

### Zhang, F., Deisseroth, K., Mishelevich, D J, and M B Schneider, “System for Optical Stimulation of Target Cells,” Application Number: 12/522,528, 2008-01-09, Publication US2010/0190229, Date 2010-07-29.

Boyden, E S, and K Deisseroth, “Light-Activated Cation Channel and Uses Thereof,” U.S. patent application Ser. No. 12/715,259 (Division of application Ser. No. 11/459,637, filed 2006-07-24) filed 2010-03-01, U.S. Publication US 2010/0234273, 2010-09-16.
Denison, T J, Kunal, P, Munns, G O, Santa, W A, Cong, P, Nielsen, C S, Norton, J D, and J G Keimel, “Optical Stimulation Therapy,” International Application Number PCT/US2010/057878 filed 2010-11-23, International Publication Number WO 2011/066320, 2011-06-03.

Unlike other forms of neuromodulation, optogenetic neuromodulation allows either excitation or inhibition (sometimes referred to as optogenetic stabilization) to be accomplished by directly depolarizing or hyperpolarizing neural membranes by shining different wavelengths of light on membranes that have had microbial opsins incorporated within them (e.g., by transfection). The primary opsins that have been used are Channelrhodopsin-2 (ChR2) used for excitation with the cation channel activated by blue light in the frequency range of 470 to 480 nm and Halorhodopsin (NpHR) used for inhibition with the chloride pump activated by amber light in the frequency range of 550 to 626 nm. Other opsins would work as well. The time scale is on the order of a millisecond. Stimulation mechanisms include optical stimulation by either local Light-Emitting Diodes (LEDs) or fiber-optic transmission of the light. Denison et al. describe variation of optical intensity/amplitude, pulse width, pulse shape, pulse rate, burst frequency, burst rate, burst width, and optical fiber configuration (e.g., as described, combination of optical fibers used with what intensity and wavelengths). An external programmer is also described.

Other forms of neuromodulation such as Deep-Brain Stimulation (DBS) or Transcranial Magnetic Stimulation (TMS) depend on the frequency of stimulation to differentiate between excitation and inhibition.

In some cases collection of signals generated by the stimulation is described. Zhang et al. describe collecting light emitted by membrane responding to the optical stimulation and Denison et al. describe collecting local bioelectric signals or temperature. In the latter, the delivery of light is adjusted using closed-loop control based on determining the patient therapeutic state based on sensing of bioelectric (or thermal) signals.

Zhang et al. describers using containment material that supports release of vectors into aqueous solution (e.g., using dehydrated or water-soluble materials such as gelatins (e.g., Matrigel from BD Biosciences)) to impact target cells. One mechanism mentioned was containment of photosensitive cells in synthetic mesh with dendrites and axons protruding and acting on other cells.

Because of the utility of optogenetics in the neuromodulation of deep-brain and other neural structures, it would be both logical and desirable to apply it to the treatment of clinical conditions. While clinical conditions that might be amenable to treatment with optogenetics have been listed, with rare exceptions, instructions have not been specific enough for actual treatment. It is thus desirable to provide specific instructions for such treatments so they can be actually implemented practically.

### SUMMARY OF THE INVENTION

It is the purpose of this invention to provide methods and systems using optogenetic neuromodulation for the treatment of clinical conditions or achievement of a physiological state. This includes specific sets of targets, up regulation or down regulation of the targets. Such neuromodulation can produce acute effects or Long-Term Potentiation (LTP) or Long-Term Depression (LTD). Included are of control of optical intensity/amplitude, pulse width, pulse shape, pulse rate, burst frequency, burst pattern, burst rate, burst width, and optical-fiber or other light-generation configuration (e.g., as described, combination of optical fibers used with what intensity and wavelengths) including through the stimulation of incorporated opsins in the target neural membranes accomplishing up-regulation and/or down-regulation.

Multiple targets can be neuromodulated singly or in groups to treat various clinical conditions. To accomplish the treatment, in some cases the neural targets will be up regulated and in some cases down regulated, depending on the given neural target. Targets have been identified by such methods as PET imaging, fMRI imaging, and clinical response to Deep-Brain Stimulation (DBS) or Transcranial Magnetic Stimulation (TMS). Location of one or more specific targets in a specific patient will involve imaging, perhaps augmented by coordinates from a brain atlas. Also included are patient feedback, operator feedback, or automated feedback (e.g., from image analysis or externally measured physiological responses).

### BRIEF DESCRIPTION OF THE DRAWINGS

FIGS. 1A and 1B show the conditions to be treated or physiological results achieved, the targets too be optogenetically neuromodulated and whether the given targets would usually be up regulated or down regulated.

FIG. 2 shows a block diagram of the control circuit.

FIG. 3 shows a block diagram of feedback control by the patient.

FIG. 4 shows a block diagram of feedback control by either the operator or automatically though external sensing such as externally measured physiological response or image analysis.

### DETAILED DESCRIPTION OF THE INVENTION

It is the purpose of this invention to provide methods and systems for neuromodulation of deep-brain and other neural targets in mammals using optogenetics to treat clinical conditions or achievement of a physiological state by identification of specific target sets for each given condition or state and whether the targets would be up regulated or down regulated. Such neuromodulation systems can produce applicable acute or long-term effects. The latter occur through Long-Term Depression (LTD) or Long-Term Potentiation (LTP) via training Included is control of optical intensity/amplitude, pulse width, pulse shape, pulse rate, burst frequency, pulse pattern, burst rate, burst width, and optical-fiber configuration including through the stimulation of incorporated opsins in the target neural membranes accomplishing up-regulation and/or down-regulation.

A critical element to the clinical application of optogenetic neuromodulation is the identification of suitable targets or target sets. In the current application these are identified along with whether a given target in a clinical-application context would be up regulated or down regulated. Patent applications the prior art are incomplete. For example, in Schneider, M B, Mishelevich, D J, and K Deisseroth, “System for Optical Stimulation of Target Cells,” U.S. patent application Ser. No. 11/651,422 filed 2007-01-09, Publication Number US 2008/0085256, 2008-04-10, a number of clinical applications are noted including heart failure, muscular dystrophies, diabetes, pain, cerebral palsy, paralysis, depression, schizophrenia, Parkinson's Disease, brain injuries, cardiac dysrhythmias, and muscle spasm but no targets are mentioned so practical application is wanting. In addition, no such clinical applications are claimed.

In Boyden, E S, and K Deisseroth, “Light-Activated Cation Channel and Uses Thereof,” U.S. patent application Ser. No. 12/715,259 (Division of application Ser. No. 11/459,637, filed on 2006-07-24) filed 20120-03-01, U.S. Publication US 2010/0234273, the specification for Boyden and Deisseroth refers to stimulation of peripheral nerves to activate dorsal-column-medial lemiscus neurons to suppress painful C-fiber responses, stimulation of retinal ganglion cells to mitigate against the loss of rod or cone loss dues to retiniatis pigmentosa or macular degeneration, treatment of depression with neuromodulation of anterior and/or subgenu cingulate cortex and to anterior limb of internal capsule, treatment of chronic pain by stimulation of the anterior and/or dorsal cingulate cortex, treatment of obesity by stimulation of ventromedial nucleus of the thalamus, treatment of obsessive compulsive disorder (OCD) by stimulation of anterior limb of internal capsule subthalamic nuclei of the thalamus, treatment of addiction by stimulation of the nucleus accumbens and septum, treatment of Alzheimer's by stimulation of the hippocampus, and treatment of Parkinson's Disease by stimulation of the subthalamic nuclei and/or Globus Pallidus.

In terms of claims in Boyden and Deisseroth, with respect to claim 2, while the conditions to be treated include depression, obsessive compulsive disorder, addiction, and Parkinson's disease, a set of neural targets is listed (anterior cingulate cortex, subgenu cingulate cortex, dorsal cingulate cortex, subthalamic nuclei, nucleus Accumbens, septum, hippocampus, and the Globus Pallidus), there is no way these are applicable to all conditions so it is clear no specific treatment method is invented. With respect to claim 7, the neural model for Parkinson's Disease is mentioned, but no specific target is mentioned. Whether one would up regulate or down regulate to accomplish therapy is not covered. The current invention does include those details as well designating different sets of targets.

With respect to the patent applications of Zhang et al., target tissues are included composed of cells that are electrically excitable, including neurons, skeletal, cardiac, smooth muscle, and insulin-secreting pancreatic beta cells. Diseases mentioned include heart failure, depression, schizophrenia, paralysis, pain, diabetes, paralysis, and cerebral palsy, and muscular dystrophies, Parkinson's disease, brain injuries, diabetes, muscle spasms, and cardiac dysrhythmias. The patent application also describes drug screening by seeing the impact of drugs on the excitatory or inhibitory response to optical stimulation. A specific application is inhibition of the Subthalamic Nucleus (STN) and the Globus Pallidus interna (GPi) for the treatment of Parkinson's Disease.

Denison et al. mentions Brain, Spinal Cord, Cardiac Therapy (pacing, cardioversion, defibrillation), Gastrointestinal (obesity, motility disorders dyspepsia), Pelvic Floor Therapy (pain, urinary or fecal incontinence therapy) or cranial nerve therapy (e.g., relieve occipital neuralgia, facial pain, migraine headaches, etc.). Examples of potential targets listed are pedunculopontine nucleus (PPN), thalamus, zona inserta, fiber tracts, lenticular fasciculus, ansa lenticularis, and Field of Forel (thalamic fasciculus). While a number of conditions are listed to which optogenetic neuromodulation might be applied and a list of potential targets supplied, with the exception of relief of migraine headaches via stimulation of the visual cortex and relief of Parkinson's disease, spasticity, and dystonia via stimulation of the subthalamic nucleus (STN), control of atrial fibrillation via neuromodulation of the atrio-ventricular node, and the conversion of Ventricular Tachycardia by stimulation of the atrium, and the pedunculopontine nucleus (PPN), relief of epilepsy by stimulating epileptic foci, the patent application does not address specifics of which targets are related to which conditions, thus allowing practical therapy to be rendered. The missing relationships would be required to make practical therapy possible, but were not. In addition specific clinical applications were not claimed. The patent application does included closed-loop control (e.g., in Parkinson's Disease or epilepsy) of neuromodulation by sensing local bioelectric signals (or temperature).

Aravanis et al. does include examples of clinical-condition-relevant targets and whether targets would be simulated or inhibited, but what is specified is incomplete and/or different than the current invention. For ease of comparison, the difference in approach for each conditions or physiological result is included in this specification at the points where the approach to each treatment or achievement of a physiological response is presented.

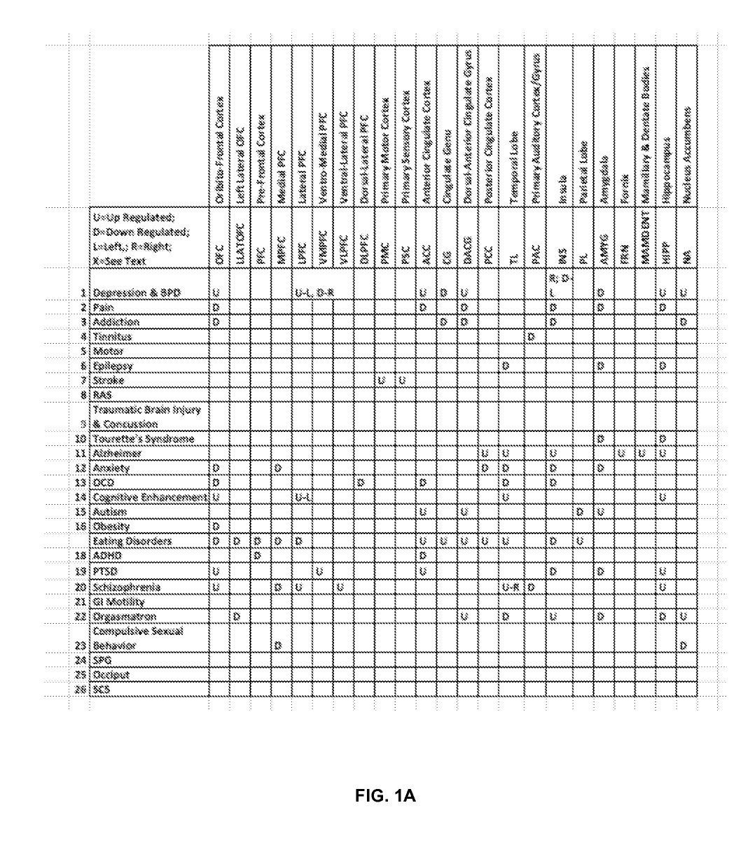

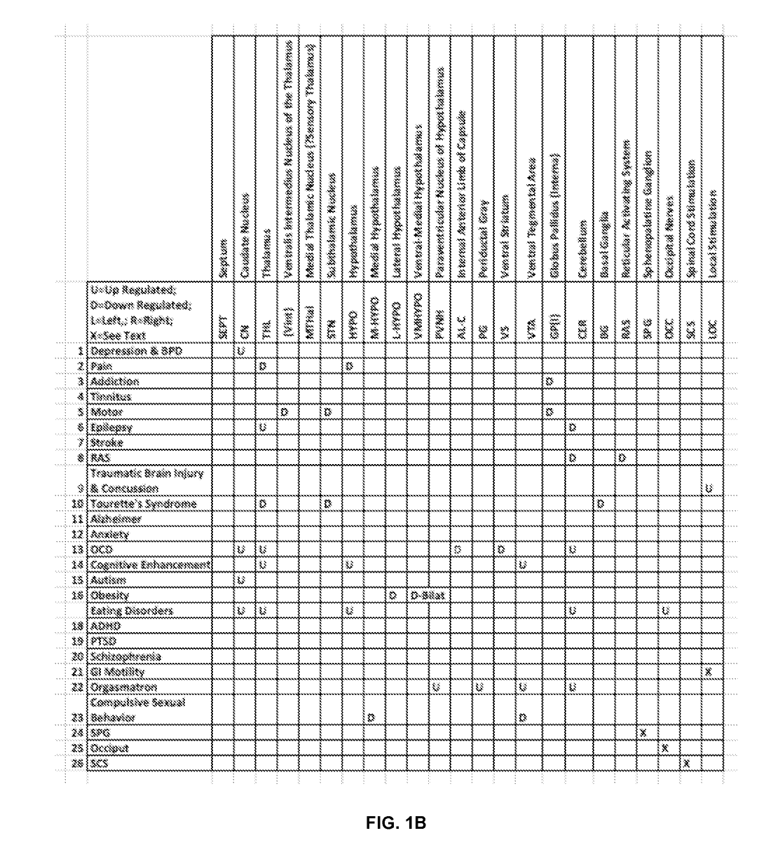

The optogenetic neuromodulation approaches to clinical conditions to be treated or the physiological results to be achieved follow. In each case statements are included as to whether a given target would be up regulated (excited) or down regulated (inhibited or stabilized). This is to be interpreted as to be “usually be up regulated” and “usually be down regulated” since this may vary for individual patient/circumstance. These aspects of the invention are shown in FIG. 1. While constant application of optogenetic neuromodulation is performed, in other cases there can be retraining of neural pathways and the optogenetic neuromodulation can be performed intermittently or potentially turned off period. In the case of the latter it could be that the implanted neuromodulator would be removed.

Depression, Bipolar Disorder, and Mood Disorders:

The Left Prefrontal Cortex would be up regulated (George, M. S., Wassermann, E. M., Williams, W. A., Callahan A., Ketter, T. A., Basser, P., Hallett, M., and R. M. Post, “Daily repetitive transcranial magnetic stimulation (rTMS) improves mood in depression,” Neuroreport 1995; 6:1853-1856), the Right Prefrontal Cortex down regulated (Menkes, D. L., Bodnar, P., Ballesteros, R. A., and M. R. Swenson, “Right frontal lobe slow frequency repetitive transcranial magnetic stimulation (SF r-TMS) is an effective treatment for depression: a case-control pilot study of safety and efficacy,” J Neurol Neurosurg Psychiatry 1999; 67:113-115), Orbito-Frontal Cortex (OFC) (Lee, Seong, et al., 2007 (Lee, B. T., Seong, Whi Cho, Hyung, Soo Khang, Lee. B. C., Choi I. G., Lyoo, I. K., and B. J. Ham, “The neural substrates of affective processing toward positive and negative affective pictures in patients with major depressive disorder,” Prog Neuropsychopharmacol Biol Psychiatry. 2007 Oct. 1; 31(7):1487-92. Epub 2007 Jul. 5)) would be up regulated, the Anterior Cingulate Cortex (ACC) would be up regulated (Lee, Seong, et al., 2007), the Subgenu Cingulate (Johansen-Berg, H., Gutman, D. A., Behrens, T. E., Matthews, P. M., Rushworth, M. F., Katz, E., Lozano, A. M., and H. S. Mayberg, “Anatomical connectivity of the subgenual cingulate region targeted with deep brain stimulation for treatment-resistant depression,” Cereb Cortex. 2008 June; 18(6):1374-83. Epub 2007 Oct. 10.) down regulated, the Right Insula (Lee, Seong, et al., 2007) up regulated, the left Insula (Lee, Seong, et al., 2007) down regulated, the Nucleus Accumbens (Hauptman, J. S., DeSalles, A. A., Espinoza, R., Sedrak, M., and W. Ishida, “Potential surgical targets for deep brain stimulation in treatment-resistant depression.,” Neurosurg Focus. 2008; 25(1):E3) up regulated, the Caudate Nucleus (Lee, Seok et al, 2008 (Lee, B. T., Seok, J. H., Lee, B. C., Cho, S. W., Yoon, B. J., Lee, K. U., Chae, J. H., Choi, I. G., and B. J. Ham, “Neural correlates of affective processing in response to sad and angry facial stimuli in patients with major depressive disorder,” Prog Neuropsychopharmacol Biol Psychiatry. 2008 Apr. 1; 32(3):778-85. Epub 2007 Dec. 23.)) up regulated, the Amygdala (Lee, Seong, et al., 2007) down regulated, and the Hippocampus (Lee, Seok et al, 2008) up regulated. The specific targets and/or whether the given target is up regulated or down regulated, can depend on the individual patient and relationships of up regulation and down regulation among targets, and the patterns of stimulation applied to the targets. In some cases neuromodulation will be bilateral and in others unilateral. Aravanis et al. is different in that it includes the Orbito-Frontal Cortex (and the Orbito-Medial Cortex) without stating whether the target was to be up regulated or down regulated, does not include the Lateral Pre-Frontal Cortex (uses the Dorsal-Lateral Pre-Frontal Cortex instead), mentions the Anterior Cingulate Cortex without saying whether is to be up regulated or down regulated, does not include the Dorsal Anterior Cingulate Cortex or the Insula, mentions the Amygdala and Nucleus Accumbens without saying whether up or down regulated, and does not include the Caudate Nucleus.

Pain:

The primary targets for pain are the Rostral Anterior Cingulate Cortex (RACC) and the Dorsal Anterior Cingulate Gyrus (DACG). In other embodiments other neural targets known to be involved in pain processing such as the orbitofrontal cortex, insula, amygdalae, thalamus, hypothalamus, and hippocampus can be neuromodulated combined with or substituted for the Rostral Anterior Cingulate Cortex (RACC) or the Dorsal Anterior Cingulate Gyrus (DACG). Aravanis et al. is different in that it does not include the Oribito-Frontal Cortex, Dorsa Anterior Cingulate Gyrus, the Insula, Amygdala, Hippocampus, Thalamus, and Hypothalamus as relevant targets, for the Anterior Cingulate Cortex it does not specify up regulation or down regulation, and it adds the Cingulate Genu.

Addiction:

Targets have been identified by such methods as PET imaging, fMRI imaging, and clinical response to Transcranial Magnetic Stimulation (TMS). Currently, the Orbito-Frontal Cortex (OFC) (Wang Z., Faith, M., Patterson, F., Tang, K., Kerrin, K., Wileyto, E. P., Detre, J. A., and C. Lerman, “Neural substrates of abstinence-induced cigarette cravings in chronic smokers,” J. Neurosci. 2007 Dec. 19; 27(51):14035-40), the Dorsal Anterior Cingulate Gyrus (DACG) (Goldstein, Rita Z., Alia-Kleina, Nelly, Tomasia, D., Honorio Carrillo, J., Maloneya, T., Patricia A. Woicika, Wanga, R., Telang, F., and Nora D. Volkow, “Anterior cingulate cortex hypoactivations to an emotionally salient task in cocaine addiction,” PNAS, 106(23): 9453-9458, Jun. 9, 2009, www.pnas.org_cgi_doi_{—}10.1073_pnas.0900491106), the Insula (Naqvi, N. H., Rudrauf, D., Damasio, Hanna, and A. Bechara, “Damage to the Insula Disrupts Addiction to Cigarette Smoking” (abstract). Science 315 (5811): 531-4), January 2007), Nucleus Accumbens (Di Chiara, G., Bassareo, V., Fenu, S., De Luca, M. A., Spina, L., Cadoni, C., Acquas, E., Carboni, E,. Valentini, V., and D. Lecca, “Dopamine and drug addiction: the nucleus accumbens shell connection,” Neuropharmacology. 2004; 47 Suppl 1:227-410, and the Globus Pallidus (Miller, J. M., Vorel, S. R., Tranguch, A. J., Kenny, E. T., Mazzoni, P., van Gorp, W. G., and H. D. Kleber, “Anhedonia After a Selective Bilateral Lesion of the Globus Pallidus,” Am J Psychiatry 163:786-788, May 2006, doi: 10.1176/appi.ajp.163.5.786) would all be down regulated. Aravanis et al. does not include the Orbito-Frontal Cortex, the Cingulate Genu, the Dorsal Anterior Cinglate Gyrus, and the Globus Pallidus interna as relevant targets, it calls from up regulation rather than down regulation of the Nucleus Accumbens, and adds the Septum, and the Medial Hypothalamus.

Tinnitus:

The primary auditory cortex is essentially in the same region as Brodmann areas 41 and 42. It is located in the posterior half of the superior temporal gyrus and also dives into the lateral sulcus as the transverse temporal gyri. Neuromodulation of the Primary Auditory Cortex (PAC) using repetitive Transcranial Magnetic Stimulation (rTMS) for a week at 1 Hz. demonstrated the elimination or reduction of tinnitus in over 50% of the patients (Rossi S, De Capua A, Ulivelli M, et al. Effects of repetitive transcranial magnetic stimulation on chronic tinnitus: a randomized, crossover, double blind, placebo controlled study. J Neurol Neurosurg Psychiatry. 2007; 78(8):857-863). Kleinjung et al. (Kleinjung T, Steffens T, Londero A, Langguth B, “Transcranial magnetic stimulation (TMS) for treatment of chronic tinnitus: clinical effects,” Prog Brain Res. 2007; 166:359-67) located the target that they used for TMS stimulation by looking at areas of increased metabolic activity demonstrated in chronic tinnitus patients by PET imaging using 18F deoxyglucose (FDG) and fusing the images with structural MRI scans to obtain anatomic correlations. Aravanis et al. does not address tinnitus.

Motor Disorders:

Targets useful for the treatment of motor disorders have been demonstrated with the results of Deep Brain Stimulation (DBS) therapy. Typically DBS is used when the quality of life of the patient decreases to an unsatisfactory level on medications of the side effects of those medications become severe. Areas that control movement are the subthalamic nucleus (STN), the ventralis intermedius nucleus of the Thalamus (Vint), and the Globus Pallidus interna (GPi). For Parkinson's disease (PD), those structures are the subthalamic nucleus (STN) or Globus Pallidus interna (GPi). Aravanis et al. does not include the Ventral Intermediate Nucleus of the Thalamus as a relevant motor-disorder target.

Epilepsy:

Targets have been identified by such methods as PET imaging, fMRI imaging, and clinical response to Deep-Brain Stimulation (DBS) or Transcranial Magnetic Stimulation (TMS). Targets for treating Epilepsy have been identified such as the Hippocampus, Temporal Lobe, Thalamus, and the Cerebellum. Most targets are identified through evaluating the effect of Deep-Brain Stimulation (DBS) (Boon P, Raedt R, de Herdt V, Wyckhuys T, and K Vonck, “Electrical stimulation for the treatment of epilepsy,” Neurotherapeutics. 2009 April; 6(2):218-27. Other potential targets are the Amygdala, Dentate Nucleus, and Mamillary Body. Aravanis et al. addresses up regulation of the Cingulate Genu to disrupt incipient seizures and applying down regulation to specific excitable regions, does not address the Temporal Lobe, Amygdala, Hippocampus and Cerebellum, and for the Thalamus prescribes down regulation as opposed to up regulation.

Stroke:

Treatment would typically involve up regulation of the Primary Motor Cortex (M1) and the Primary Sensory Cortex, as applicable. All or part of the motor cortex can be damaged by a stroke and other areas may be damaged by ischemic or hemorrhagic stroke as well. Typically the edges (peripheral margins) of an area impacted by a stroke are viable and neuromodulation of these edges can mitigate against further loss of tissue acutely. In the longer term, neuromodulation of this viable tissue can foster post-stroke rehabilitation. Sensory defects can appear with damage to the Primary Sensory Cortex. Wernicke's aphasia can result from damage to Wernicke's area in the Superior Temporal Gyrus and benefit by up regulation using optogenetic neuromodulation. Other areas sometimes impacted are Broca's area, the posterior limb of internal capsule, basis pontis, thalamus, and corona radiata, all of which can benefit from up regulation. One consideration is the possible combination with Tissue Plasmin Activator or aspirin although are important implications for potential bleeding in conjunction with the surgical implantation of optogenetic neuromodulation devices. Aravanis et al. addresses stroke in terms of neuromodulation of local tissues to promote neuron growth rather than for direct physiological impacts.

Vegetative State/Control of State of Level of Consciousness:

This involves up regulation or down regulation of the Reticular Activating System (RAS) and the Cerebellum as applied for a variety of clinical purposes such as reversibly putting a patient to sleep or waking them up (for example, for the purpose of anesthesia) or reversibly putting a patient into a coma (for example for the purpose of protecting or rehabilitating the brain of the patient after a stroke or head injury). Aravanis et al. does not address this arena.

Traumatic Brain Injury & Concussion:

Neuromodulation is applied to up regulate or down regulate local damaged tissue areas (e.g., up regulate to stimulate activity and down regulate to mitigate against seizure activity) and to up regulate the Thalamus. Aravanis et al. addresses Traumatic Brain Injury in terms of neuromodulation of local tissues to promote neuron growth.

Tourette's Syndrome:

Targets for treating Tourette's Syndrome have been identified such as the hippocampus and amygdala (Peterson, B S, Choi, H A, Hoa, X, Amat, J A, Zhu, H, Whiteman, R, Liu, J, Xu, D, and R Bansal, “Morphologic features of the amygdala and hippocampus in children and adults with Tourette syndrome,” Arch Gen Psychiatry. 2007 November; 64(11):1281-91), both of which would be down regulated. Other potential targets are the thalamus, sub-thalamic nuclei, and basal ganglia. Aravanis et al. does not include the Amygdala, Hippocampus, and Subthalamic Nucleus as relevant targets.

Alzheimer's Disease:

For treatment of Alzheimer's Disease and other dementias, primary neural targets are the Hippocampus (Henneman W J, Sluimer J D, Barnes J, van der Flier W M, Sluimer I C, Fox N C, Scheltens P, Vrenken H, and F Barkhof, “Hippocampal atrophy rates in Alzheimer disease: added value over whole brain volume measures,” Neurology. 2009 Mar. 17; 72(11):999-1007), Posterior Cingulate Gyrus (PCG) (Awad M, Warren J E, Scott S K, Turkheimer F E, and R J Wise, “A common system for the comprehension and production of narrative speech,” J. Neurosci. 2007 Oct. 24; 27(43):11455-64), Temporal Lobe (Zhang Y, Londos E, Minthon L, Wattmo C, Liu H, Aspelin P, and L O Wahlund, “Usefulness of computed tomography linear measurements in diagnosing Alzheimer's disease,” Acta Radiol. 2008 February; 49(1):91-7), Formix (Ringman J M, O'Neill J, Geschwind D, Medina L, Apostolova L G, Rodriguez Y, Schaffer B, Varpetian A, Tseng B, Ortiz F, Fitten J, Cummings J L, and G Bartzokis G, “Diffusion tensor imaging in preclinical and presymptomatic carriers of familial Alzheimer's disease mutations,” Brain. 2007 July; 130(Pt 7):1767-76. Epub 2007 May 23), Mamillary Body (Copenhaver B R, Rabin L A, Saykin A J, Roth R M, Wishart H A, Flashman L A, Santulli R B, McHugh T L, and A C Mamourian, “The formix and mammillary bodies in older adults with Alzheimer's disease, mild cognitive impairment, and cognitive complaints: a volumetric MRI study,” Psychiatry Res. 2006 Oct. 30; 147(2-3):93-103. Epub 2006 Aug. 22), and Dentate Gyrus (Bramham C R, “Control of synaptic consolidation in the dentate gyrus: mechanisms, functions, and therapeutic implications,” Prog Brain Res. 2007; 163:453-71), all of which are to be up regulated. An example of a non-Alzheimer's dementia is Temporal-Frontal dementia related to the Anterior Cingulate and the Frontoinsular cortex (Seeley, W., Carlin, Danielle A., Allman, J, Macedo, M., Bush, Clarissa, Miller, B. and S. J. DeArmond, “Early Frontotemporal Dementia Targets Neurons Unique to Apes and Humans,” Ann Neurol 2006; 60:660-667; Published online Dec. 22, 2006 in Wiley InterScience, (www.interscience.wiley.com). DOI: 10.1002/ana.21055). Aravanis et al. does not include the Posterior Cingulate Cortex, the Temporal Lobe, the Insula, the Formix, and the Mammillary and Dentate Bodies as relevant targets and with respect to the Posterior Cingulate Cortex does not indicate whether it would be up regulated or down regulated.

Anxiety:

Neural targets central to anxiety are the Posterior Cingulate Cortex (PCC) (Zhao X H, Wang P J, Li C B, Hu Z H, Xi Q, Wu W Y, and X W Tang X W, “Altered default mode network activity in patient with anxiety disorders: an fMRI study,” Eur J. Radiol. 2007 September; 63(3):373-8. Epub 2007 Apr. 2), Amygdala (Milad M R and SL Rauch S L, “The role of the orbitofrontal cortex in anxiety disorders,” Ann N Y Acad Sci. 2007 December; 1121:546-61. Epub 2007 Aug. 14), Insula (Reiman E M, Raichle M E, Robins E, Mintun M A, Fusselman M J, Fox P T, Price J L, and K A Hackman, “Neuroanatomical correlates of a lactate-induced anxiety attack,” Arch Gen Psychiatry. 1989 June; 46(6):493-500), Orbito-Frontal Cortex (OFC) (Schienle A, Schäfer A, Hermann A, Rohrmann S, and D Vaitl, “Symptom provocation and reduction in patients suffering from spider phobia: an fMRI study on exposure therapy,” Eur Arch Psychiatry Clin Neurosci. 2007 December; 257(8):486-93. Epub 2007 Sep. 27). Other targets include the Medical Prefrontal Cortex (MPFC) and the Temporal Lobe. Depend on specific patients and relationships among the targets. Aravanis et al. does not include the Medial Pre-Frontal Gyrus, the Posterior Cingulate Cortex, or the Insula as relevant targets.

Obsessive Compulsive Disorder (OCD):

Targets for treating obsessive-compulsive disorder have been identified through means of Deep Brain Stimulation (DBS) (for example, Baker K B, Kopell B H, Malone D, Horenstein C, Lowe M, Phillips M D, and A R Rezai, “Deep brain stimulation for obsessive compulsive disorder: using functional magnetic resonance imaging and electrophysiological techniques: technical case report,” Neurosurgery. 2007 November; 61(5 Suppl 2):E367-8; discussion E368) and imaging studies (for example, Nakao T, Nakagawa A, Nakatani E, Nabeyama M, Sanematsu H, Yoshiura T, Togao O, Tomita M, Masuda Y, Yoshioka K, Kuroki T, and S Kanba, “Working memory dysfunction in obsessive-compulsive disorder: a neuropsychological and functional MRI study,” J Psychiatr Res. 2009 May; 43(8):784-91. Epub 2008 Dec. 10). The former identified the Head the Caudate (ipsilateral to the stimulated Ventral Striatum, if stimulated), Medial Thalamus, Anterior Cingulate Cortex (ACC), Ventral Striatum, and Cerebellum (contralateral to the Ventral Striatum, if stimulated). The latter identified the Orbito-Frontal Cortex (OFC), the right Dorsal Lateral Prefrontal Cortex (DLPFC), the left Superior Temporal Gyrus, the left Insula, and the Cuneus. Yucel et al. (Yücel, M, Wood, S J, Fornito, A, Riffkin, Judith, Velakoulis D, and C Pantelis, “Anterior cingulate dysfunction: Implications for psychiatric disorders?,” J Psychiatry Neurosci. 2003 September; 28(5): 350-354) is an example of another article discussing the role of the Anterior Cingulate Cortex. The OFC, ACC, Ventral Striatum, Insula, Cuneus, and Dorsal-Lateral PFC, Superior Temporal Lobe are down regulated and the Head of the Caudate, (Medial) Thalamus, and Cerebellum are up regulated. Aravanis et al. does not include the Orbito-Frontal Cortex, the Dorsal Lateral Pre-Frontal Cortex, the Temporal Lobe, the Insula, the Caudate Nucleus, the Thalamus, the Ventral Striatum, and the Cerebellum as relevant targets, and adds the Cingulate Genu.

Cognitive Enhancement:

Multiple targets can be neuromodulated singly or in groups for cognitive enhancement. Cognitive enhancement can be applied for two broad purposes, first that involving cognitive enhancement where cognitive faculties have been diminished (e.g., Alzheimer's Disease, Alzheimer's Disease, Parkinson's disease, Creutzfeld-Jacob disease, Attention Deficit Hyperactivity Disorder, dementia and stroke) and second that involving enhancement of cognitive function in a normal individual. Thus the type of application of cognitive enhancement can be to abnormal function or normal function. It is to be noted that some question the ethics of using means to enhance cognitive function in a person with normal cognition (Mendelsohn, D. Lipsman, N. and M. Bernstein, “Neurosurgeons' Perspectives on Psychosurgery and Neuroenhancement: a Qualitative Study at One Center,” J. Neurosurg. 2010 December; 113(6): 1212-8. Epub 2020 Jun. 4). Neural targets identified include the Ventral Tegmentum, the Hypothalamus, the Central Thalamus (Shirvalkar, P., Seth, M., Schiff, N. D., and D. G. Herrera, “Cognitive Enhancement with Central Thalamic Electrical Stimulation,” PNAS Nov. 7, 2006 vol. 103 no. 45 17007-17012), the anterior cortex, and the Fronto-Temporal Lobe. Lazano and Mayberg (U.S. Patent Application 2006/0201090, “Method of Treating Cognitive Disorders Using Neuromodulation”) describe an invention using electrical and/or chemical stimulation of a variety of targets for the treatment of a variety of conditions but are non-specific about what target is related to what condition and do not cover cognitive enhancement in normal individuals.

Snyder and his colleagues have studied the impact of TMS used to inhibit anterior areas (including the Fronto-Temporal Lobe) of the brain on normal subjects (Snyder, A., Bossomaier, T., and D. J. Mitchell, “Concept Formation: ‘Object’ Attributes Dynamically Inhibited from Conscious Awareness,” Journal of Integrative Neuroscience 3(1), 31-46, 2004 and Snyder, A. W., Mulcahy, E., J. L., Taylor, et al., “Savant-Like Skills Exposed in Normal People by Suppressing the Left Fronto-Temporal lobe. Journal of Integrative Neuroscience 2(2), 149-158, 2003). They found that ability to spell check was improved and that drawing style was changed to a more concrete style. They postulated this was due to reducing top-down semantic control. This could be related to work of Miller et al. (Miller, B. L., Ponton, M., Benson, D. F., Cummings, J. L., & I. Mena, “Enhanced artistic creativity with temporal lobe degeneration,” Lancet, 348, 1744-1755, 1996) who looked at previously normal patients with Fronto-Temporal Lobe Dementia who demonstrated emergence of new artistic skills along with their dementia, although attributing this to a different neural mechanism. Miller and colleagues attributed this to deterioration of the Orbito-Frontal Lobe and Anterior Temporal Lobe resulting in an impact on visual systems related to perception whose inhibition was decreased. One application of the invention is to provide a tune up to concretize learning for a student studying for a test.

Calendar calculation can be used to identify targets for cognitive enhancement. For example, Boddaert et al. (Boddaert, N., Barthelemy, C., Poline, J. B., Samson, Y., Brunelle, F., & M. Zilbovicius, M., “Autism: Functional brain mapping of exceptional calendar capacity,” British Journal of Psychiatry, 187, 83-86, 2005) used PET imaging compared calendar calculation to rest in an adult with autism. This demonstrated activation of brain regions usually associated with memory (Left Hippocampus, Left Frontal Cortex, and Left Middle Temporal Lobe). Aravanis et al. does not include the Lateral Pre-Frontal Cortex, the Temporal Lobe, the Thalamus, the Hypothalamus, and the Ventral Tegmental Area as relevant targets and does add the Parietal Lobe.

Autism Spectrum Disorder:

For treatment of Autism Spectrum Disorder, primary neural targets are the Parietal Lobe, Amygdala, Anterior Cingulate Gyrus, and Caudate Nucleus. The Parietal Lobe was identified by Wong et L. (Wong T K, Fung P C, Chua S E, and G M McAlonan, “Abnormal spatiotemporal processing of emotional facial expressions in childhood autism: dipole source analysis of event-related potentials,” Eur J. Neurosci. 2008 July; 28(2):407-16), Gomot et al. (Gomot M, Belmonte M K, Bullmore E T, Bernard F A, and S. Baron-Cohen, “Brain hyper-reactivity to auditory novel targets in children with high-functioning autism,” Brain. 2008 September; 131(Pt 9):2479-88. Epub 2008 Jul. 31) and Shafritz et al. (Shafritz K M, Dichter G S, Baranek G T, and A Belger, “The neural circuitry mediating shifts in behavioral response and cognitive set in autism,” Biol Psychiatry. 2008 May 15; 63(10):974-80. Epub 2007 Oct. 4). The Amygdala was identified by Pinkham et al. (Pinkham A E, Hopfinger J B, Pelphrey K A, Piven J, and DL Penn, “Neural bases for impaired social cognition in schizophrenia and autism spectrum disorders”, Schizophr Res. 2008 February; 99(1-3):164-75. Epub 2007 Nov. 28). The Anterior Cingulate Gyrus was identified by Haznedar et al. (Haznedar M M, Buchsbaum M S, Wei T C, Hof P R, Cartwright C, Bienstock C A, and E. Hollander, “Limbic circuitry in patients with autism spectrum disorders studied with positron emission tomography and magnetic resonance imaging,” Am J. Psychiatry. 2000 December; 157(12):1994-2001) and the Caudate Nucleus by Degirmenci et al. (Degirmenci B, Miral S, Kaya G C, Iyilikçi L, Arslan G, Baykara A, Evren I, and H. Durak, “Technetium-99m HMPAO brain SPECT in autistic children and their families,” Psychiatry Res. 2008 Apr. 15; 162(3):236-43. Epub 2008 Mar. 4). A subset of these targets would also work and other targets may be discovered as well. In the application of the therapeutic ultrasound, the Parietal Lobe would be down regulated, and the Anterior Cingulate Cortex (ACC), the Amygdala, and Caudate Nucleus up regulated. Aravanis et al. does not address Autism as an as a condition for treatment.

Obesity:

For treatment of obesity, primary neural targets are the Orbito-Frontal Cortex (OFC) that is to be down regulated, the Ventromedial Hypothalamus (VMH) that is to be down regulated bilaterally, and the Lateral Hypothalamus (LH) that is to be down regulated. Aravanis et al. does not include the Orbito-Frontal Cortex as a relevant target and does include the Hypothalamus. With respect to the Ventral-Medial Hypothalamus, it describes up regulating rather than down regulating it.

Eating Disorders:

Targets for treating the eating disorder Anorexia Nervosa have been identified such the Anterior Cingulate Cortex (ACC) and the Pre-Frontal Cortex (PFC). In patients with Anorexia Nervosa, the volume of the Anterior Cingulate Cortex (ACC) is decreased (Mühlau M, Gaser C, Ilg R, Conrad B, Leibl C, Cebulla M H, Backmund H, Gerlinghoff M, Lommer P, Schnebel A, Wohlschlager A M, Zimmer C, and S Nunnemann, “Gray matter decrease of the anterior cingulate cortex in anorexia nervosa,” Am J. Psychiatry. 2007 December; 164(12):1850-7). Regional cerebral blood flow have shown that the Pre-Frontal Cortex (PFC) is involved (Matsumoto R, Kitabayashi Y, Narumoto J, Wada Y, Okamoto A, Ushijima Y, Yokoyama C, Yamashita T, Takahashi H, Yasuno F, Suhara T, and K Fukui, “Regional cerebral blood flow changes associated with interoceptive awareness in the recovery process of anorexia nervosa,” Prog Neuropsychopharmacol Biol Psychiatry. 2006 Sep. 30; 30(7):1265-70. Epub 2006 Jun. 14). Another target identified in the study was the Posterior Cingulate Cortex (PCC). Among the targets identified in patients with Bulimia are the Anterior Cingulate Cortex (ACC), and Dorsal Anterior Cingulate Gyrus (DACG) and the Caudate Nucleus (Marsh, R, Steinglass, J, Gerber, A J, O'Leary, K G, Wang, Z, Murphy, D, Walsh, B T, and B S Peterson, “Deficient Activity in the Neural Systems That Mediate Self-regulatory Control in Bulimia Nervosa,” Arch Gen Psychiatry. 2009; 66(1):51-63). Other targets related to Bulimia are superior temporal gyrus/insula (relative deactivation relative to normal), the Pre-Frontal Cortex, and the Caudate Nucleus (Brooks, S J, O'Daly, O G, Uher, R, Friederich, H-C, Giampietro, V, Brammer, M, Williams, S C R, Schiöth, H B, Treasure, J, and I C Campbell, “Differential Neural Responses to Food Images in Women with Bulimia versus Anorexia Nervosa,” PLoS One. 2011; 6(7): e22259, Published online 2011 Jul. 20. doi: 10.1371/journal.pone.0022259). In summary, areas involved are the Frontal and Pre-Frontal Areas (Orbito-Frontal Cortex, Left Lateral Orbito-Frontal Cortex, Pre-Frontal Cortex, Medial Pre-Frontal Cortex, Lateral Pre-Frontal Cortex) and the Insula, all of which would be down regulated, the Cingulate Areas (Anterior Cingulate Cortex, Cingulate Genu, Dorsal Anterior Cingulate Gyrus, Posterior Cingulate Cortex), the Temporal Lobe, the Parietal Lobe, the Caudate Nucleus, the Thalamus, the Hypothalamus, the Cerebellum, and the Occipital Nerve, all of which would be up regulated. Aravanis et al. does not include the Dorsal Anterior Cingulate Gyrus and the Occipital Nerves as relevant targets and with respect to the Pre-Frontal components and the Insula, they are down regulated instead of up regulated.

Attention Deficit Hyperactivity Disorder (ADHD):

Targets for treating Attention Deficit Hyperactivity Disorder have been identified such as the Pre-Frontal Cortex (PFC) and Anterior Cingulate Cortex (ACC) based on imaging studies (Fassbender C and J B Schweitzer, “Is there evidence for neural compensation in attention deficit hyperactivity disorder? A review of the functional neuroimaging literature,” Clin Psychol Rev. 2006 August; 26(4):445-65. Epub 2006 Feb. 24). These targets have also been identified based on the decreased volumes of those structures in ADHD (Seidman L J, Valera E M, Makris N, Monuteaux M C, Boriel D L, Kelkar K, Kennedy D N, Caviness V S, Bush G, Aleardi M, Faraone S V, and J Biederman J, “Dorsolateral prefrontal and anterior cingulate cortex volumetric abnormalities in adults with attention-deficit/hyperactivity disorder identified by magnetic resonance imaging,” Biol Psychiatry. 2006 Nov. 15; 60(10):1071-80. Epub 2006 Jul. 28). Other targets are the Superior Parietal Lobe, Medial Temporal Lobe, Basal Ganglia/Striatum, Caudate Nucleus, Superior Colliculus, and the Cerebellum. Aravanis et al. does not address ADHD as an as a condition for treatment.

Post-Traumatic Stress Disorder (PTSD):

For treatment of PTSD, primary neural targets are the Amygdala, Hippocampus, Anterior Cingulate Cortex, Orbito-Frontal Cortex, and the Insula. An additional target can be the Ventro-Medial Pre-Frontal Cortex and others may be discovered as well. One consideration is that PTSD may involve dysfunction of the Hypothalamic, pituitary-adrenal axis involving the Hippocampus, Amygdala, and Pre-Frontal Cortex (PFC) as in Ruiz et al. (Ruiz J E, Barbosa Neto J, Schoedl A F, and M F Mello M F, “Psychoneuroendocrinology of posttraumatic stress disorder,” Rev Bras Psiquiatr. 2007 May; 29 Suppl. 1:S7-12.). In the application of the therapeutic ultrasound, the hyperactive Amygdala would be down regulated, the Anterior Cingulate Cortex (ACC) up regulated, the Orbito-Frontal Cortex (OFC) up regulated, the Hippocampus up regulated, and the Insula down regulated. If the Ventro-Medical Pre-Frontal Cortex were targeted it would be up regulated.

The Amygdala, Anterior Cingulate Cortex, Orbito-Frontal Cortex, and the Hippocampus targets were identified in Jatzko et al. (Jatzko A, Schmitt A, Kordon A, and D F Braus D F, “Neuroimaging findings in posttraumatic stress disorder: review of the literature,” Fortschr Neurol Psychiatr. 2005 July; 73(7):377-91.). Involvement of the Amygdala, and Anterior Cingulate Cortex plus addition of the Insula was identified in Liberzon et al. (Liberzon I, Britton J C, and K L Phan K L, “Neural correlates of traumatic recall in posttraumatic stress disorder,” Stress. 2003 September; 6(3):151-6.). Aravanis et al. does not include the Orbito-Frontal Cortex, the Ventro-Medial Pre-Frontal Cortex, the Anterior Cingulate Cortex, and the Insula as relevant targets. For the Hippocampus it describes down regulating rather than up regulating it.

Schizophrenia:

Targets for treating Schizophrenia have been identified such as Orbito-Frontal Cortex (OFC) (Nakamura M, Nestor P G, Levitt J J, Cohen A S, Kawashima T, Shenton M E, and R W McCarley, “Orbitofrontal volume deficit in schizophrenia and thought disorder,” Brain. 2008 January; 131(Pt 1):180-95. Epub 2007 Dec. 3) which would be up regulated since it involves a decrease in volume in schizophrenia, the Primary Auditory Cortex which would be down regulated, the Medial Pre-Frontal Cortex (MPFC) (Taylor S F, Welsh R C, Chen A C, Velander A J, and I Liberzon, “Medial frontal hyperactivity in reality distortion,” Biological Psychiatry. 2007 May 15; 61(10):1171-8. Epub 2007 Apr. 16) which would be down regulated since it is hyperactive in schizophrenia, the Dorsal-Lateral Pre-Frontal Cortex (DLPFC) (Karlsgodt K H, Sanz J, van Erp T G, Bearden C E, Nuechterlein K H, and T D Cannon, “Re-evaluating dorsolateral prefrontal cortex activation during working memory in schizophrenia,” Schizophrenia Research 2009 March; 108(1-3):143-50. Epub 2009 Feb. 3) which would be up regulated because it is hypoactive in schizophrenia, the Ventral-Lateral PFC (VLFPC) (Pinkham A E, Hopfinger J B, Pelphrey K A, Piven J, and D L Penn, “Neural bases for impaired social cognition in schizophrenia and autism spectrum disorders,” Schizophrenia Research 2008 February; 99(1-3):164-75. Epub 2007 Nov. 28) which would be up regulated because it is hypoactive on the left in schizophrenia, the Entorhinal region of the Temporal Lobe (Baiano M, Perlini C, Rambaldelli G, Cerini R, Dusi N, Bellani M, Spezzapria G, Versace A, Balestrieri M, Mucelli R P, Tansella M, and P Brambilla, “Decreased entorhinal cortex volumes in schizophrenia,” Schizophrenia Research 2008 July; 102(1-3):171-80. Epub 2008 Jan 14) which would be up regulated on the right side because of a decrease in volume in schizophrenia, and the Hippocampus (N Kuroki, M Kubicki, P G Nestor, D F Salisbury, H-J Park, J J Levitt, S Woolston, M Frumin, M Niznikiewicz, C-F Westin, S E Maier, R W McCarley, and M E Shenton, “Formix Integrity and Hippocampal Volume in Male Schizophrenic Patients,” Biological Psychiatry, Volume 60, Issue 1, Pages 22-31, 1 Jul. 2006) which would be up regulated because of bilateral reduction of volume in schizophrenia. Aravanis et al. does not include the Hippocampus and the Temporal Lobe as relevant targets, and adds the Insula.

GI Motility:

Gastrointestinal activity can be modified by optogenetic neuromodulation. The results can be assessed objectively by myoelectric activity, measurement of pressure changes, and detection of motion, say by movement of accelerometers. Such sensors can be built in to a neuromodulation device passing through the GI tract, can be placed in a separate sensing device passing through or inserted into the GI tract, or for myoelectric signals can be detected by sensors external to the body such as myoelectric signals captured by electrodes placed on the skin. A variety of gastrointestinal organs can be neuromodulated (e.g., esophagus, stomach, small intestine, cecum, ascending colon, transverse colon, descending colon, sigmoid colon, rectum, and anus). An additional target is the vagal nerve. Signals indicating level of gastrointestinal motility (e.g., by electrogastroenterogram, electromyography, internal electrodes, internal pressure sensors, microphones, imaging, or other suitable means) can be detected and patient feedback can be used (say with the patient seating on a commode) to adjust the characteristics of the optogenetic neuromodulation.

Orgasmatron and Anhedonia:

As to one or more targets, up-regulation is to be applied to the Dorsal Anterior Cingulate Gyrus, the Insula, the Cerebellum, the Paraventricular Nucleus of the Hypothalamus, the Nucleus Accumbens, The Ventral Tegmental Area (VTA) and the Periductal Grey, and down regulation to the Left Lateral Orbito-Frontal Gyrus, the Amydala, the Temporal Lobe, and the Hippocampus. Aravanis et al. does not address the Orgasmatron or Anhedonia.

Compulsive Sexual Behavior:

As to targets (e.g., Hilton D L, Watts C. Pornography addiction: A neuroscience perspective. Surg Neurol Int 2011; 2:19), the Medial Pre-Frontal Cortex, Nucleus Accumbens, Hypothalamus, and Ventral Tegmental Area are down regulated. Aravanis et al. does not include the Medial Pre-Frontal Cortex, the Hippocampus, and the Ventral Tegmental Area as relevant targets.

Sphenopalatine Ganglion (SPG):

Electrical stimulation of the Sphenopalatine Ganglion (and other elements of the autonomic nervous system) has been used in the treatment of migraine and cluster headaches (Pless (B. D. Pless, “Method and Device for the Treatment of Headache,” U.S. Patent Application Pub. No. 2009/0276005). Such stimulation can also include neuromodulation of the Sphenopalatine Nerve and the Vidian Nerve and can treat not only pain, but also nausea and vomiting as well. Any of the targets would be up regulated in this application. Aravanis et al. does not address the Spenopalatine Ganglion as a region for treatment.

Occiput:

Transcranial Magnetic Stimulation and electrical stimulation of occipital nerves has been used in the treatment of headaches (Burns, B., Watkins, L., and P. Goadsby, “Treatment of medically intractable cluster headache by occipital nerve stimulation: long-term follow-up of eight patients,” The Lancet, Volume 369, Issue 9567, Pages 1099-1106, 31 Mar. 2007) and optogenetic neuromodulation is an effective alternative. The Occipital Nerve targets would be up regulated in this application. Aravanis et al. does not address the Occiput as a region for treatment.

Spinal Cord Stimulation (SCS):

In this clinical application, optogenetic neuromodulation of the Spinal Cord and its connections is used to treat certain types of pain. For the treatment of neuropathic pain it has been shown the Spinal Cord Stimulation using electrodes suppresses hyperexcitability of the neurons via alteration of dorsal horn neurochemistry including the release of serotonin, Substance P, and GABA. For treatment of ischemic pain, it has been suggested that the oxygen supply is restored via the possibilities of sympathetic stimulation and/or vasodilation. The usual mode will be up regulation. Aravanis et al. describes local stimulation and inhibition plus down regulating selected tracts to suppress motor tics as well as using up regulation to stimulation of new tracts via employment of stem cells.

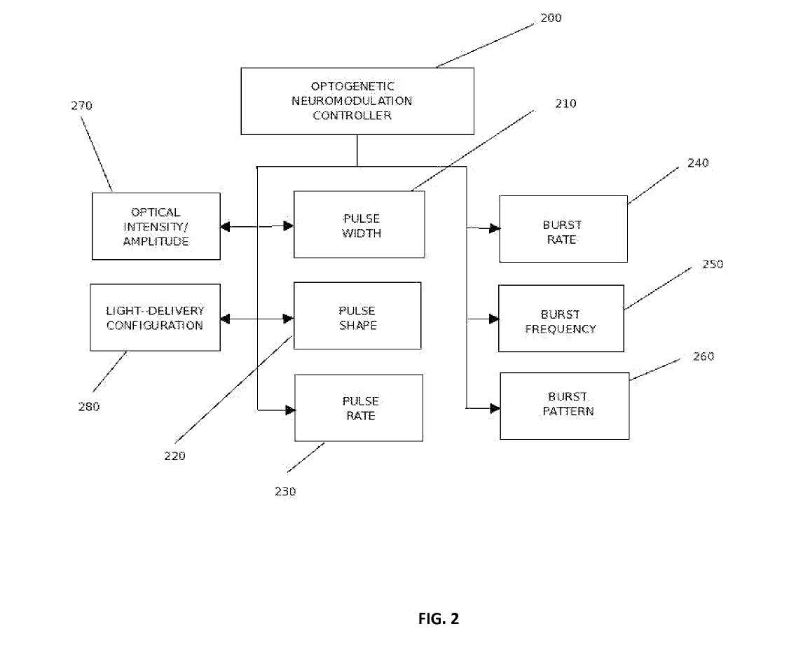

FIG. 2 shows an embodiment of a control circuit. The light output is under overall control of Optogenetic Neuromodulation Controller 200, with pulse characteristics of Pulse Width 210, Pulse Shape 220, and Pulse Rate 230, burst characteristics of Burst Rate 240, Burst Frequency 250, and Burst Pattern 260 along with Optical Intensity/Amplitude 270, and Light-Delivery Configuration 280.

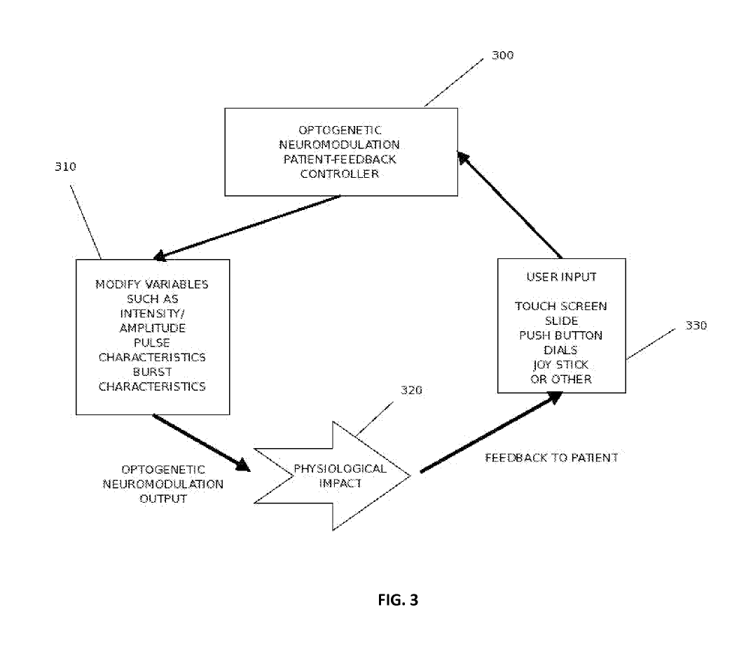

FIG. 3 shows a block diagram of the use of Patient-Mediated Feedback mechanism is applied to adjust the optogenetic neuromodulation variables. The output is under control of Optogenetic Neuromodulation Patient-Feedback Controller 300 that instructs modification of variables 310 (e.g., Intensity/Amplitude, Pulse Characteristics, Burst Characteristics) with the Optogenetic Neuromodulation Output having a Physiological Impact 320 which provides Feedback to the Patient (e.g., less or more pain or anxiety) so that the Patient can provide User Input 330 (using such input mechanisms such as one or more of a Touch Screen, Slide, Push Button, Dials, Joy Stick or similar) to Optogenetic Neuromodulation Patient-Feedback Controller 300. Denison et al. covers feedback from internal bioelectric or thermal signals, but not patient feedback.

In another embodiment, a feedback mechanism is applied based on an external feedback mechanism such as operator feedback, physiological monitoring, functional Magnetic Resonance Imaging (fMRI), Positive Emission Tomography (PET) imaging, video-electroencephalogram (V-EEG), acoustic monitoring, thermal monitoring, or other external. Such use of ancillary monitoring or imaging is optional. An aspect of the current invention is that optogenetic neuromodulation device elements that might influence MRI imaging are MRI compatibility. In embodiments where concurrent imaging is performed, the device is constructed of non-ferrous material. Denison et al. covers feedback from internal bioelectric or thermal signals, not external signals.

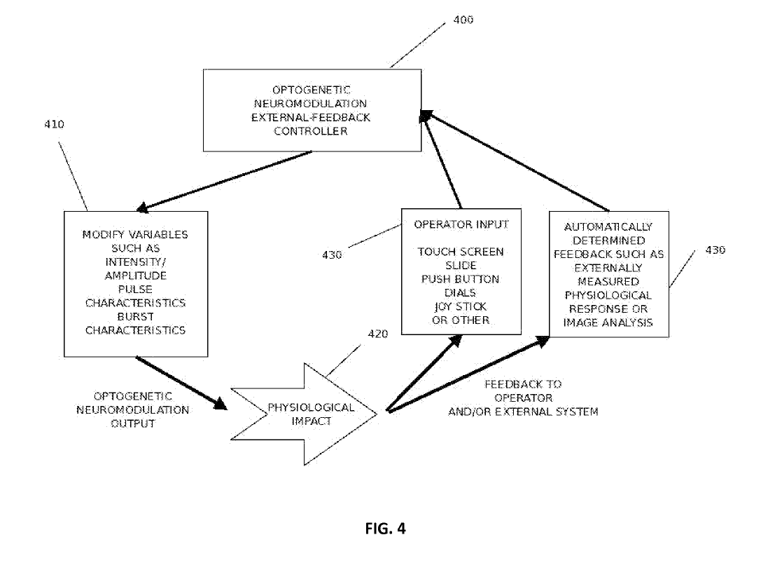

FIG. 4. Shows a block diagram of the use of External Feedback mechanism being applied to adjust the optogenetic neuromodulation variables. The output is under control of Optogenetic Neuromodulation External-Feedback Controller 400 that instructs modification of variables 410 (e.g., Intensity/Amplitude, Pulse Characteristics, Burst Characteristics) with the Optogenetic Neuromodulation Output having a Physiological Impact 420 the result of which provides Feedback to the Operator (e.g., change in movement or Visual Analog Score) or External System (e.g., measured movement with transducer or image analysis) so that the Operator can provide Operator Input 430 (using such input mechanisms such as one or more of a Touch Screen, Slide, Push Button, Dials, Joy Stick or similar) or the External System can provide feedback automatically determined via Externally Measured Physiological Response or Image Analysis 430 to Optogenetic Neuromodulation External-Feedback Controller 400.

In still other embodiments, other energy sources are used in combination with or substituted for optogenetic optical sources that are selected from the group consisting of Transcranial Magnetic Stimulation (TMS), Deep-Brain Stimulation (DBS), Ultrasound Neuromodulation, radiosurgery, Radio-Frequency (RF) therapy, behavioral therapy, and medications. Multi-modality neuromodulation is covered in D J Mishelevich, “Multi-Modality Neuromodulation of Brain Targets,” U.S. patent application Ser. No. 12/958,411, filed Feb. 2, 2012. Any past, present or future neuromodulation methods would be applicable.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

What is claimed is:

1. A method of neuromodulating neural structures in a living mammals using optogenetic neuromodulation, the method comprising:
inserting opsins in one or a plurality of neural targets, and
applying optogenetic neuromodulation via a control circuit,
whereby a clinical condition is alleviated or a physiological state is achieved.

2. The method of claim 1 further comprising aiming an ultrasound transducer neuromodulating neural targets in a manner selected from the group of up-regulation, down-regulation.

3. The method of claim 1 wherein the condition to be treated is selected from the group consisting of Depression, Bipolar Disorder, and Mood Disorders and the one or plurality of targets to be neuromodulated are selected from the group consisting of Orbito-Frontal Cortex (OFC), Lateral Pre-Frontal Cortex, Anterior Cingulate Cortex, Cingulate Genu, Dorsal Anterior Cingulate Gyrus, Insula, Amygdala, Hippocampus, Nucleus Accumbens, and Caudate Nucleus.

4. The method of claim 3 where, if given target included, Orbito-Frontal Cortex (OFC) is usually down regulated, Lateral Pre-Frontal Cortex is usually up regulated on the left and down regulated on the right, Anterior Cingulate Cortex usually up regulated, Cingulate Genu usually down regulated, Dorsal Anterior Cingulate Gyrus usually up regulated, Insula usually up regulated on the right and down regulated on the left, Amygdala usually down regulated, Hippocampus usually up regulated, Nucleus Accumbens usually up regulated, and Caudate Nucleus usually up regulated.

5. The method of claim 1 wherein the condition to be treated is pain and the one or a plurality of targets to be neuromodulated are selected from the group consisting of Orbito-Frontal Cortex (OFC), Anterior Cingulate Cortex, Dorsal Anterior Cingulate Gyrus, Insula, Amygdala, Hippocampus, Thalamus, and Hypothalamus.

6. The method of claim 5 where, if given target included, Orbito-Frontal Cortex (OFC), Anterior Cingulate Cortex, Dorsal Anterior, Insula, Amygdala, Hippocampus, Thalamus, and Hypothalamus, would all usually be down regulated.

7. The method of claim 1 wherein the condition to be treated is addiction and the one or a plurality of targets to be neuromodulated are selected from the group consisting of Orbito-Frontal Cortex (OFC), Dorsal Anterior Cingulate Gyrus (DACG), Insula, Nucleus Accumbens, and Globus Pallidus interna (GPi).

8. The method of claim 7 where, if given target included, Orbito-Frontal Cortex (OFC), Dorsal Anterior Cingulate Gyrus (DACG), Insula, Nucleus, Accumbens, and Globus Pallidus interna (GPi) would all usually be down regulated.

9. The method of claim 1 wherein the condition to be treated is motor disorders and the one or a plurality of targets to be neuromodulated are selected from the group consisting of Subthalamic Nucleus (STN), Globus Pallidus interna, and the ventralis intermedius nucleus of the thalamus (Vint).

10. The method of claim 9 where, if given target included, Subthalamic Nucleus (STN), Globus Pallidus interna, and the ventralis intermedius nucleus of the thalamus (Vint), would all usually be down regulated.

11. The method of claim 1 wherein the condition to be treated is stroke and the one or plurality of targets to be neuromodulated are selected from the group consisting of Primary Motor Cortex, Primary Sensory Cortex, Superior Temporal Gyrus (Wernicke's area), Broca's area, the posterior limb of internal capsule, basis pontis, thalamus, and corona radiata.

12. The method of claim 11 where, if given target included, the Primary Motor Cortex, Primary Sensory Cortex, Superior Temporal Gyrus (Wernicke's area), Broca's area, the posterior limb of internal capsule, basis pontis, thalamus, and corona radiate, would all usually be down regulated.

13. The method of claim 1 wherein the condition to be treated is pain and the one or a plurality of targets to be neuromodulated are selected from the group consisting of the Amygdala, Hippocampus, Thalamus, Subthalamic Nucleus, and Basal Ganglia.

14. The method of claim 13, if given target included, the Amygdala, the Hippocampus, the Thalamus, the Subthalamic Nucleus, and the Basal Ganglia would all usually be down regulated.

15. The method of claim 1 wherein the condition to be treated is autism spectrum disorder and the one or a plurality of targets to be neuromodulated are selected from the group consisting of Anterior Cingulate Cortex (ACC), Dorsal Anterior Cingulate Gyrus (DACG), Parietal Lobe, Amygdala, and Caudate Nucleus.

16. The method of claim 15, where, if given target included, the Anterior Cingulate Cortex (ACC), Dorsal Anterior Cingulate Gyrus (DACG), Parietal Lobe, Amygdala, and Caudate Nucleus would all usually be up regulated and the Parietal Lobe would usually be down regulated.

17. The method of claim 21 where, if given target included, the Sphenopalatine Ganglion, the Sphenopalatine Nerve, and the Vidian Nerve, would all usually be up regulated.

18. The method of claim 1, wherein the optogenetic variables controlled are one or a plurality of optical intensity/amplitude, pulse width, pulse shape, pulse rate, burst frequency, burst pattern, burst rate, burst width, and optical-fiber or other light-generation configuration.

19. (canceled)

20. The method of claim 1 wherein a feedback mechanism is applied to adjust the neuromodulation variables, wherein the feedback mechanism is selected from the group consisting of patient feedback, operator feedback, physiological monitoring, functional Magnetic Imaging (fMRI), Positive Emission Tomography (PET) imaging, video-electroencephalogram (V-EEG), acoustic monitoring, thermal monitoring, or other external.

21. The method of claim 1 wherein the condition to be treated is selected from the group consisting of pain, nausea, vomiting and the one or a plurality of targets to be neuromodulated are selected from the group consisting of the Sphenopalatine Ganglion, the Sphenopalatine Nerve, and the Vidian Nerve.
